(19) United States
(12) Patent Application Publication
(10) Pub. No.: US 2019/0298451 A1
(43) Pub. Date: Oct. 3, 2019

(54) SYSTEMS AND METHODS FOR DELIVERING TARGETED THERAPY

(71) Applicant: INTUITIVE SURGICAL OPERATIONS, INC., Sunnyvale, CA (US)

(72) Inventors: Serena H. Wong, Los Altos, CA (US); Kyle R. Miller, San Jose, CA (US)

(21) Appl. No.: 16/364,876

(22) Filed: Mar. 26, 2019

Related U.S. Application Data
(60) Provisional application No. 62/648,773, filed on Mar. 27, 2018.

(51) Int. Cl. A61B 34/10; A61B 1/05; A61B 1/00; A61B 17/32; A61B 17/00; G16H 20/40

(52) U.S. Cl. CPC A61B 34/10; A61B 1/05; A61B 1/00135; A61B 17/320068; A61B 17/00234; A61B 2017/00349; A61B 2034/105; A61B 2034/107; A61B 2017/320069; A61B 2017/003; A61B 2017/00818; G16H 20/40

(57) ABSTRACT

The systems and methods of the present disclosure are for use in a surgical procedure in which an elongate flexible medical device is utilized to create a model of patient anatomy using information from one or more of a localization sensor and an imaging sensor. Anatomical areas of interest may be identified and displayed on the model of patient anatomy, which may be used in performing a medical procedure on at least one of those areas of interest.

### CROSS-REFERENCE TO RELATED APPLICATIONS

This application claims the benefit of U.S. Provisional Application 62/648,773 filed Mar. 27, 2018, which is incorporated by reference herein in its entirety.

### FIELD

The present disclosure is directed to systems and methods for care during surgical procedures. In particular, the present disclosure is directed to accessing and providing therapy to intra-body target tissue during a surgical procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments (including surgical, diagnostic, therapeutic, or biopsy instruments) to reach a target tissue location. It would be desirable to provide a delivery system which would help guide a user to safely and accurately place surgical devices near target tissue during a medical procedure.

### SUMMARY

The embodiments of the invention are best summarized by the claims that follow the description. Consistent with some embodiments, a method is performed, by a processor, for accessing and treating a target tissue within a patient anatomy. The method comprises creating a first model of the patient anatomy using information from at least one localization sensor and at least one imaging sensor. The at least one localization sensor and the at least one imaging sensor are coupled to a medical device being delivered through the patient anatomy to the target tissue. The method also comprises receiving data identifying at least one anatomical area of interest within the patient anatomy and creating a second model based on the first model and the at least one identified anatomical area of interest. The method also comprises displaying the second model and performing a medical procedure on at least one of the anatomical areas of interest.

Consistent with some embodiments, a medical system for accessing a patient anatomy comprises a medical device including a main lumen and a localization sensor providing position of the medical device. The medical system also comprises an imaging sensor configurable to provide an image from a distal portion of the medical device and a processor in communication with the localization sensor and the imaging sensor. The processor is configured to generate a first model using information from the localization sensor and the imaging sensor, receive data for identifying anatomic features of interest within the patient anatomy, and update the first model to generate a second model including the anatomic features of interest. The medical system also includes a display for displaying at least one of the first model and the second model.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a cross-sectional view of a medical instrument system according to some embodiments.

FIG. 2B is a simplified diagram of a medical instrument with an extended medical tool according to some embodiments.

FIG. 3 is a flowchart of an example medical procedure according to some embodiments.

FIG. 4 illustrates a medical system inserted into a patient anatomy according to some embodiments.

FIG. 5 is a detailed view of a patient anatomy according to some embodiments.

FIG. 6 illustrates a medical system inserted into a patient anatomy according to some embodiments.

FIGS. 7A-7C illustrate a medical system inserted into a patient anatomy according to some embodiments.

FIG. 8 is a simplified diagram of a medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

In some instances well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian X-, Y-, and Z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

Minimally invasive procedures include introducing a minimally invasive medical instrument or elongate device through a natural orifice in a patient anatomy or through one or more surgical incisions, and navigating the instrument through patient anatomy to access anatomical target tissue for diagnosis or treatment. In one embodiment, a medical instrument or an elongate device can be used to access various areas of patient anatomy such as within a patient digestive system, respiratory system, renal system, circulatory system, and/or reproductive system. In some cases, an instrument with a working lumen can be navigated through anatomy and positioned at the anatomical target and then used as a delivery conduit for additional medical devices including surgical, diagnostic, delivery, therapeutic, and biopsy instruments. Examples of medical devices include an endoscope, an ablation device, a biopsy tool, a surgical tool, an ultrasound probe, and/or the like. The minimally invasive medical instruments and devices can be flexible or rigid and navigation of the instruments can be manually and/or robotically controlled by a user (e.g. operator, clinician, physician, etc.) under endoscopic guidance and/or under imaging guidance such as fluoroscopy.

In some cases, depending on the type of medical procedure to be performed and/or the anatomy to be navigated, it can be difficult to identify target tissue or sensitive anatomy which should be avoided. Pre-operative images (such as CT, MRI, and/or the like) can be used to create a model of the anatomy but in many cases, imaging technology may not be available, capturing pre-operative imaging data may not be a standard practice for a planned procedure, models may not adequately display the target tissue or sensitive anatomy, or anatomy may shift or changes in the anatomy may occur between the time of imaging and the time of the procedure. Thus it may be helpful to generate accurate models or maps of anatomy, plan medical procedures according to the models, and display the models with planned procedural information for guidance as an aid to diagnose, visualize and/or treat different disease states or conditions. It may also be helpful, especially in cases requiring treatment of tissue, to map a degree of overlap of successive treatment areas to allow for successive treatments down an anatomical lumen and have desired overlap of treatment areas or avoid overlap of treatment areas. For example, with use of RF ablation, no overlap is desired and can even harm the anatomical lumen causing stenosis. In the case of other types of treatments, a fixed overlap can be arranged if desired as well. Thus, with the use of position sensors in conjunction with visual confirmation, models of anatomy can be generated, medical procedures can be planned, and models can be updated based on disease state and target anatomy.

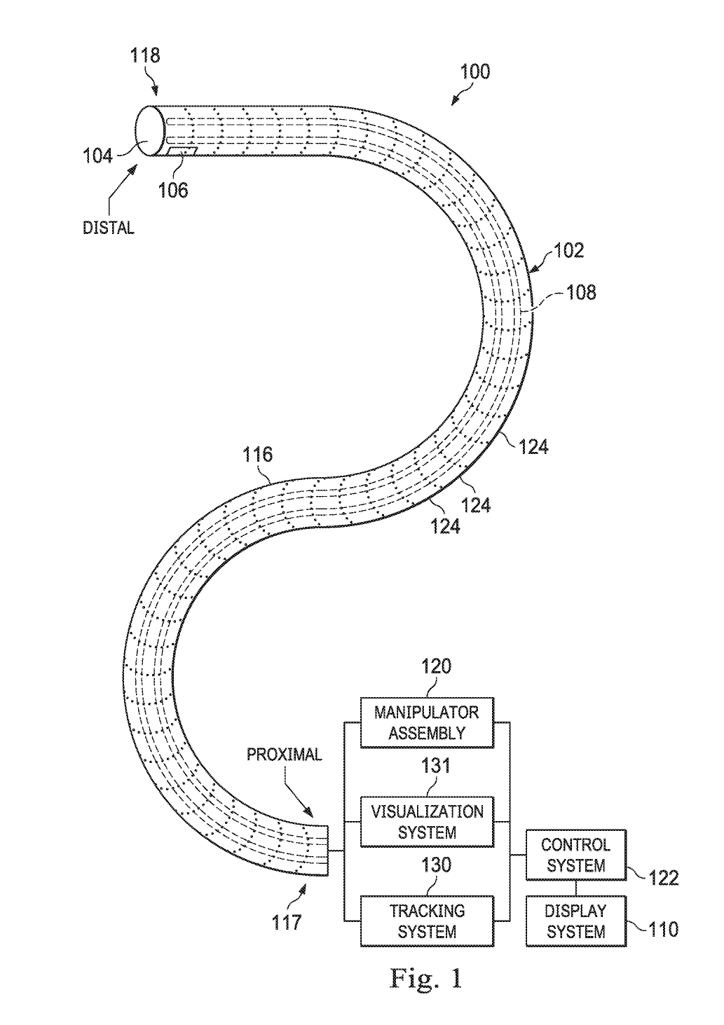

FIG. 1 is a medical system 100 in accordance with the present invention. Medical instrument system 100 includes an elongate device 102 having a flexible body 116 and a main lumen 104 (also known as a working lumen, main channel, or working channel) and a localization sensor or set of localization sensors which may be integrated into a wall of the elongate device 102. Alternatively, the localization sensor may be slideably disposed in main lumen 104 or another lumen (not shown) or otherwise integrated into the body of elongate device 102. The flexible body 116 extends between a distal end 118 and a proximal end 117. The elongate device 102 may be coupled to or in communication with a variety of systems including a manipulator assembly 120, a visualization system 131, a tracking system 130. Either directly or via at least one of the manipulator assembly 120, the visualization system 131, and/or the tracking system 130, the elongate device 102 may be coupled to or in communication with a control system 122 and a display system 110.

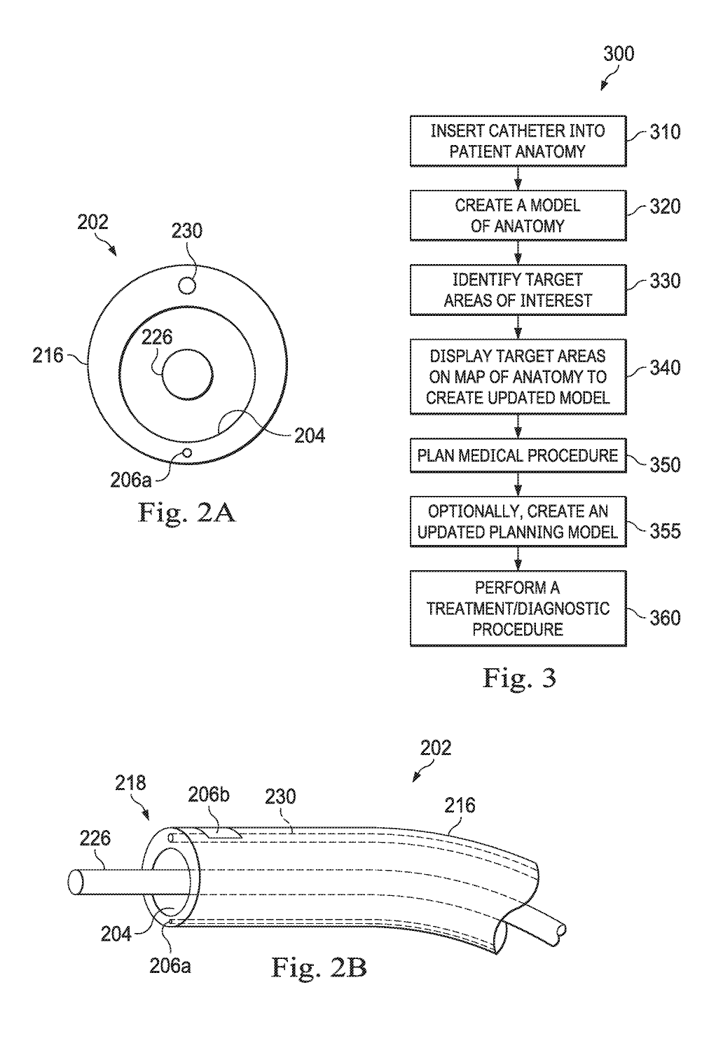

The main lumen 104, may be used as an open delivery channel for delivery of various devices (as is described below with reference to FIGS. 2A and 2B) as well as devices used to aid in steering of the elongate device 102, such as guide wires, overtubes, and/or the like. The localization sensor/s could also be integrated within another device inserted into the main lumen 104 of the elongate device 102 and used as part of the system for localization and navigation; it may be removed during treatment, especially if a treatment device includes separate localization sensor/s. Localization sensors can include sensors (e.g. a single position sensor 106, a plurality of position sensors distributed along the length of the elongate device 102, a shape sensor 108, and/or an imaging sensor) coupled to the tracking system 130 for receiving and processing sensor data and information for determining the position, orientation, speed, velocity, pose, and/or shape of distal end 118 and/or of one or more lengths along the flexible body 116. Tracking system 130 may optionally be implemented as hardware, firmware, software or a combination thereof which interact with or are otherwise executed by one or more computer processors.

Tracking system 130 may optionally track positions and/or orientations of distal end 118 and/or one or more of segments 124 of the flexible body 116 using a shape sensor 108. Shape sensor 108 may optionally include an optical fiber aligned with flexible body 116 (e.g., provided within an interior channel (not shown) or mounted externally). The optical fiber of shape sensor 108 forms a fiber optic bend sensor for determining the shape of flexible body 116. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some embodiments may employ other suitable strain sensing techniques, such as Rayleigh scattering, Raman scattering, Brillouin scattering, and Fluorescence scattering. In some embodiments, the shape of the elongate device may be determined using other techniques. For example, a history of the distal end pose of flexible body 116 can be used to reconstruct the shape of flexible body 116 over the interval of time. In some embodiments, tracking system 130 may optionally and/or additionally track distal end 118 using a position sensor system 106. Position sensor system 106 may be a component of an EM sensor system with position sensor system 106 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some embodiments, position sensor system 106 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

In some embodiments, tracking system 130 may alternately and/or additionally rely on historical pose, position, or orientation data stored for a known point of an instrument system along a cycle of alternating motion, such as breathing. This stored data may be used to develop shape information about flexible body 116. In some examples, a series of positional sensors (not shown), such as electromagnetic (EM) sensors similar to the sensors in position sensor 106 may be positioned along flexible body 116 and then used for shape sensing. In some examples, a history of data from one or more of these sensors taken during a procedure may be used to represent the shape of elongate device 102, particularly if an anatomic passageway is generally static.

FIGS. 2A and 2B illustrate is a simplified diagram of an end view and a side view respectively, of another example of an elongate device 202 including flexible body 216 having distal end 218, main lumen 204, and localization or position sensor/s 206a and 206b. Elongate device 202 may be similar in construction and function as elongate device 102 except where described herein. In some embodiments, position sensor 206a may include an optical fiber shape sensor integrated along the length of elongate device 202. In some embodiments, position sensor 206b may include a single position sensor, such as an EM sensor, optical encoder, and/or the like, as shown. In some embodiments, position sensor 206a may be positioned in a different circumferential position as 206b as illustrated in FIGS. 2A and 2B. In some embodiments, position sensor 206a may be in a substantially similar circumferential position as 206b. In some embodiments, elongate device 202 may include a plurality of position sensors 206b positioned down the length of and/or at different locations around the circumference of the elongate device 202. In some embodiments, elongate device may include a plurality of position sensors 206a and a plurality of position sensors 206b. In some embodiments, elongate device 202 includes only a single or plurality of position sensors 206a or elongate device 202 includes a single or plurality of position sensor 206b.

In another example, position sensor 206a may be included along the length of a medical instrument 226, such that position, orientation, pose, and/or shape of the medical instrument 226 can be measured and then confirmed using imaging sensor 230 embedded within a wall of elongate device 202. Elongate device 202 may include imaging sensor 230, such tool based imaging devices for providing intraoperative images, for example endoscopic cameras, endoluminal or intravascular ultrasound, OCT device, and/or the like. The elongate device 202 position and/or orientation can be determined based on information from the imaging sensor 230 and/or from position sensor(s) 206a/206b.

The medical instrument 226 may be slidably disposed within the main lumen 204 of the elongate device 202 and extended from the distal end 218. In some embodiments, medical instrument 226 may be used for procedures such as surgery, biopsy, ablation, illumination, irrigation, or suction. Medical instrument 226 can be deployed through main lumen 204 of flexible body 216 and used at a target location within the anatomy. Medical instrument 226 may include, for example, image capture probes, biopsy instruments, laser ablation fibers, ablation balloons, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. In various embodiments, medical instrument 226 is a biopsy instrument, which may be used to remove sample tissue or a sampling of cells from a target anatomic location.

Medical instrument 226 may be visualized using imaging sensor 230 (e.g., an image capture probe) that includes a distal portion with a stereoscopic or monoscopic camera at or near distal end 218 of flexible body 216 for capturing images (including video images) that are processed by an imaging system, such as visualization system 131 for display and/or provided to tracking system, such as tracking system 130 to support tracking of distal end 218 and/or a portion of flexible body 216 along the length of flexible body. The imaging sensor may include a cable coupled to the camera for transmitting the captured image data. In some examples, the imaging sensor 230 may be a fiber-optic bundle, such as a fiberscope, that couples to visualization system 131. The imaging sensor may be single or multi-spectral, for example capturing image data in one or more of the visible, infrared, and/or ultraviolet spectrums. The imaging sensor 230 can be any type of minimally invasive tool providing intraoperative images, for example endoscopic probe, an OCT probe, or ultrasound probe (endoluminal or intravascular), or include a combination of any number of endoscopic, OCT, antennas, electrodes, other electromagnetic measurement devices, and/or ultrasound probes. Target tissue and various anatomical structures can be identified visually in endoscopic camera images as well as in, e.g., ultrasound or OCT images. Tissue may be identified from as ablated or non-ablated tissue by measuring impedance of the tissue and monitoring a change in impedance or comparing impedance measurements against pre-determined impedance thresholds.

In some embodiments, the imaging sensor 230 may be slideably disposed through main lumen 204, slideably disposed within a secondary lumen offset from main lumen 204, fixed within the secondary lumen, integrated into a wall of the elongate device 202, or disposed external to elongate device 202. Alternatively, medical instrument 226 may itself be the image capture probe. Medical instrument 226 may be advanced from the opening of main lumen 204 to perform the procedure and then retracted back into the channel when the procedure is complete. Medical instrument 226 may be removed from a proximal end of flexible body 216 or from another optional instrument port (not shown) along flexible body 216. In some embodiments flexible body 216 is not flexible but rigid or semi-rigid.

In some embodiments, a separate sheath, steerable or non-steerable, is deployed over elongate device 202. In some embodiments, the sheath is a passive sheath with an integrated optical camera or vision probe. This provides additional functionality for the device and leaves main lumen 204 available for other uses. In some embodiments, the passive sheath couples to the elongate body using a locking mechanism or keying mechanism, e.g. a proximal locking mechanism, a distal locking mechanism, keying of the passive sheath and elongate body along the length or at a distal section of sheath and elongate body. In alternative embodiments, the passive sheath may couple to the elongate body using a keyed interface along the length or at a distal end portion of the passive sheath or the elongate body.

In some examples, as described in detail below, the imaging sensor alone or in combination with other components of the medical instrument system 100 may include one or more mechanisms for cleaning one or more lenses of the imaging sensor when the one or more lenses become partially and/or fully obscured by fluids and/or other materials encountered by the distal end of the imaging sensor. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Publication No. WO/2016/025465 filed Aug. 11, 2016 disclosing “Systems and Methods for Cleaning an Endoscopic Instrument”; U.S. patent application Ser. No. 15/508,923 filed Mar. 5, 2017 disclosing “Devices, Systems, and Methods Using Mating Catheter Tips and Tools”; and U.S. patent application Ser. No. 15/503,589 filed Feb. 13, 2017 disclosing “Systems and Methods for Cleaning an Endoscopic Instrument,” each of which is incorporated by reference herein in its entirety. The imaging system may be implemented as hardware, firmware, software or a combination thereof which interact with or are otherwise executed by one or more computer processors, which may include the processors of a control system.

In some examples, the elongate device 202 is used to accurately navigate to target tissue. Once present at the target tissue, the medical instrument 226 may be delivered through the main lumen 204 of the elongate device 202 or coupled to the elongate device 202 and then used for diagnostics, visualization, and/or treatment. The medical instrument 226 may include an imaging device (endoscopic camera, ultrasound transducer, etc.) and/or a treatment device.

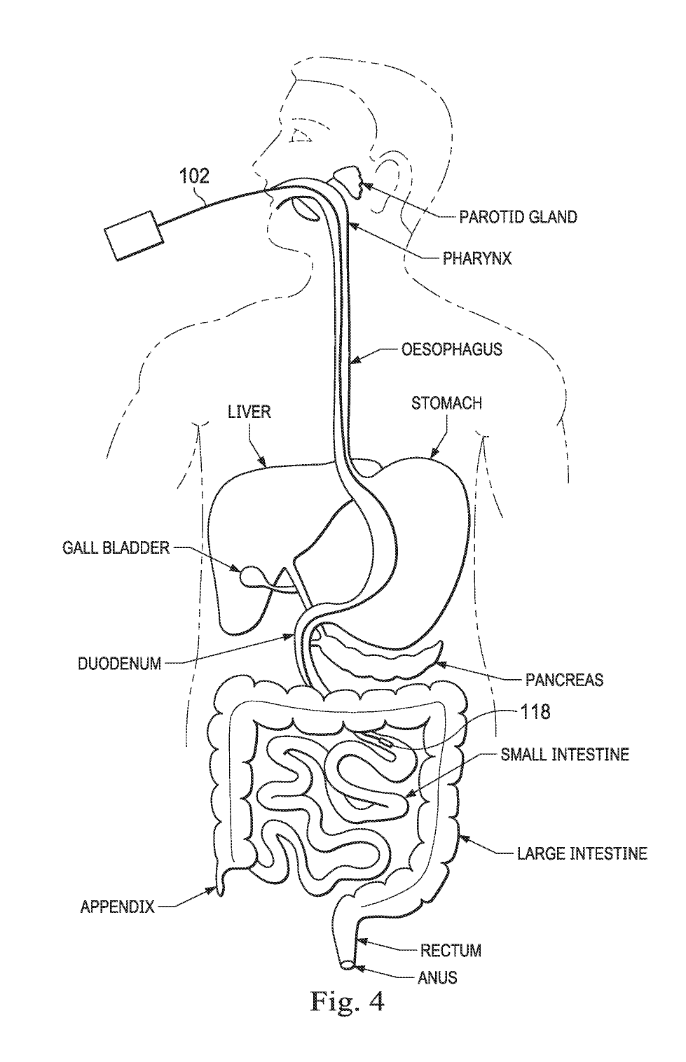

FIG. 3 is a flowchart of an example medical procedure 300 in accordance with the present invention. Medical procedure 300 may generate a model of patient anatomy, wherein the model may be used to plan areas of treatment, and/or used for treatment of the target tissue. At process 310, an elongate device such as elongate device 102, is introduced into anatomy to be navigated towards an anatomical target. The elongate device 102 can be used to access various areas of patient anatomy such as within a patient digestive system, respiratory system, renal system, and/or reproductive system, to diagnose, visualize and/or treat different disease states or conditions. In one embodiment, elongate device 102 may be inserted into a natural orifice in a patient anatomy or through one or more surgical incisions and navigated to the anatomical target. In one example, the surgical incision could be made to provide direct access to the target anatomy.

At process 320, an anatomic model, or map of patient anatomy can be created. In one embodiment, the model may be generated from data collected as elongate device 102 is navigated through an anatomical path towards the anatomical target. The data can be provided by position, localization, and imaging sensors such as shape sensor 108, position sensor 106, and imaging sensor 230 coupled to elongate device 102. This provides for real time localization, orientation, and/or position of a distal portion of the flexible body as well as the shape of the flexible body and images of internal surfaces of lumens, vessels, and/or organs. In some embodiments, a processor coupled to or provided with tracking system 130 may collect position and/or localization data representing the distal end 118 of the elongate device 102, thereby providing a centerline of the anatomy. The model may then be built including diameters of the of the anatomy by using either standard anatomical human data, using elongate device 102 and localization sensing to touch anatomical walls to determine volume, and/or data from imaging sensor 230 where diameter can be determined using image-based methods.

In some embodiments, elongate device 102 may be routed within an internal region, and the distal end 118 of the elongate device 102 may be positioned past the target tissue. Then a shape sensor, such as shape sensor 108 or a plurality of position sensors 106 positioned down a length of the flexible body 116, can be utilized to measure a partial or full shape of a length of the elongate device 102. The model of the area surrounding the target tissue may be rendered based on the partial or full shape of the elongate device 102, data gathered during navigation of elongate device 102, imaging data collected during navigation of elongate device 102 (e.g. endoscopic or ultrasound data), or any combination of all data.

In some embodiments, the model is initially generated using a generic anatomic model of average human anatomy, possibly chosen based on some heuristics such as patient weight, height, sex and age. In another embodiment, the initial model is generated from pre-operative scans from imaging data obtained from a CT, pet CT, MRI, DICOM, ultrasound or fluoroscopic images. In some cases, the pre-operative scans are taken weeks prior to a procedure so that anatomy can shift so imaging data may not accurately display all anatomical areas of interest, depending on the quality of the imaging equipment and the type of tissue or anatomical structure of interest. Thus, the initial model may then be altered, supplemented, or merged with data collected while the elongate body is navigated through anatomy, e.g. positional/location data gathered during navigation, endoscopic camera data correlated to positional data, and/or intravascular or endoluminal ultrasound data, to more accurately display the patient anatomy as previously described above.

At process 330, specific anatomical target areas of interest can be identified within the model generated in process 320. The areas may include anatomical structures within the body to be used as landmarks to be used during navigation of the device; areas identified for treatment, such as lesions, tumors, or diseased tissue; sensitive structures or anatomy to be avoided during treatment, which may be located in close proximity to the treatment areas; and/or artificial locations such as a previously placed markers within the tissue.

In some embodiments, the identification of landmarks can be user defined, such as by visually identifying the landmark using, for example a live endoscopic view or under live fluoroscopic guidance, and having the operator press a button when the elongate device is adjacent to or touching a landmark. The user can provide input to identify they type of landmark which can be saved in a computer processor. Software can compensate from a distance from the distal tip of the catheter/camera/scope to the landmark given a known focal length of the camera and the visual appearance of the landmark. In some embodiments, identification of the landmark can be vision based, where the image (e.g. the endoscopic image) is identified automatically, such as comparing the image to a library of images of anatomical features or can be visually identified by the approximate location and shape of the anatomical feature.

In some embodiments, ultrasound may be used to determine depth tissue of interest, such as lesions, tumors, and/or diseased tissue such as hyperplastic tissue. An ultrasound transducer integrated into the elongate device or treatment device or integrated into a probe delivered through the elongate device may be used to capture ultrasound data. With localization sensors provided within the elongate device, the location of the ultrasound image can be correlated to the location of the elongate device and an exact location of the tissue of interest as well as the depth of the tissue of interest can be determined.

At process 340, the target areas of interest identified in process 330 may then be displayed or rendered on the model of the anatomy generated from process 320 to create an updated model. In some embodiments, if ultrasound is used to identify depth of target tissue, the target tissue can be displayed at a measured location within the model and at a detected depth based on ultrasound data. In some embodiments, the model is a 3D model so the location and depth of the tissue target can be accurately displayed on the model and highlighted with a specific color, shade, hue, and/or transparency to visibly distinguish from healthy tissue.

At process 350, the updated model may be used to plan a medical procedure including determining a navigational path to an anatomic or ablation target and/or creating a treatment plan. The medical procedure plan can then be saved within a computer processor and/or used to provide additional detail or further update the updated model generated during process 340.

In one embodiment, the treatment plan can include type of treatment including biopsy, diagnosis, dissection, delivery of chemicals, ablation, and/or the like. Treatment plans can include determining treatment parameters such as treatment location, size of treatment area, treatment depth, number of treatments, and spacing of treatments. In some embodiments, treatment zones can be planned so that they are spaced in a particular fashion or pattern, such as helically or circumferentially. In one example, a treatment plan can be created to ablate an anatomical target, such as a tumor or nodule. Accordingly, the treatment plan can specify location of ablative energy delivery, size and depth of ablation zones, number of ablation zones, and/or an ablation pattern.

In some embodiments, power settings can be established at varied levels based on distance to sensitive anatomy, type of tissue being treated, and/or status of treatment. A power setting can be chosen based on depth of the diseased tissue. Power can be varied if ultrasound has identified deeper diseased tissue in some areas. Power can be scaled from a maximum power to zero, or a threshold distance from sensitive tissue can be established where power is set to a particular amount, such as a minimum safe level or turned off and set to zero for safety. In some embodiments, power can be reduced further away from the central area of target tissue to be treated to treat or ablate a margin around target tissue. In some embodiments, circumferential ablations may be of alternating depths, i.e. power is increased and decreased for adjacent ablations to minimize the chance of stenosis of vessels. In some embodiments, power can be decreased as ablation continues, e.g. as tissue becomes more desiccated, tissue is burned, and/or tissue cells are killed. In some embodiments, other types of variable power ablation patterns may be created.

The plan for the medical procedure can include locating sensitive areas and displaying locations which may require protection during a treatment procedure. For example, locations for deployment of a protective device can be determined and saved as part of the medical procedure plan. Locations can be identified and displayed where substances, such as saline or water, can be applied to cool and protect the sensitive areas.

In some embodiments, while the model is being generated from data collected as the flexible device is routed through anatomy as previously described, the centerline can be saved and displayed in the model as a navigation path to the anatomical target. The navigation path can be used if the elongate device is removed from patient anatomy and the operator needs to access the anatomical target in a follow up procedure or using a different medical device.

In some embodiments, the treatment plan and/or updated treatment plan can be automatically created based on the model or updated model, and empirical data of ablation size, depth, and power requirements correlated with tissue type and type of anatomical target (e.g. tumor). In other embodiments, the treatment plan can be manually created by the operator using the model for guidance. In some embodiments, power settings and locations for ablations may be determined manually by looking at the model and determining if e.g. lesser or greater ablation is needed in certain areas or a certain ablation pattern is needed. In some embodiments, a combination of software defined and manual determinations are made, e.g. the software sets an ablation pattern with power settings and the user alters the plan. In alternative embodiments, the treatment plan including power settings can be created as a combination of automatic and user input selection of treatment parameters.

Optionally, at a process 355, one or more indicators of the target tissue, the treatment locations, the power settings, the ablation pattern, the landmarks, the structures to treat or avoid are displayed on, included within, or overlaid on the updated model generated in process 340 to create an updated planning model. The medical plan can be displayed as an instructions on a display, as indicators overlaid on the anatomic model generated during process 340, and/or as updates to the anatomic model. For example, using display system 110 shown in FIG. 1, visual instructions can be provided indicating location of target tissue, power levels for ablation, duration of ablation, warning of proximity to sensitive anatomy, etc. Instructions can be audible or haptically provided. Indicators can be overlaid on the anatomic model showing location and depth of diseased tissue or indicating location and depth of desired ablation lesions (i.e. lesions created within tissue after applying ablative energy). The instructions and/or indicators can be highlighted within the current updated model by being displayed in a different color, transparency, hue, size, etc.

Using the procedure plan determined in process 350, a treatment/diagnostic procedure can be performed in process 360. Real time navigational guidance may be displayed within the updated anatomic model. The navigational guidance may include a rendered image of the elongate device or medical device represented within the anatomic model. In order to display the rendered image of the elongate device in the anatomic model correlated to a real time pose of the actual elongate device within the patient anatomy, the elongate device should be registered to the anatomic model. In some embodiments, the model is built from data, information, or images collected using the visualization device so that the model is inherently registered to the device since the visualization device (or elongate body coupled to the visualization device) includes localization sensors. In some embodiments, the model is built from data collected during insertion of the device within anatomy, where data points measured using localization, e.g. shape sensing are used to build the model. Therefore, the model and the device are inherently registered. In some embodiments, an initial model is generated from pre-operative data and the device is navigated through anatomy, collecting positional data points and identifying target areas of interest, e.g. identifying landmarks using a camera. The collection of the target area such as the landmark can also be correlated with a position or orientation of the camera. Therefore a system can use the positional data and identified landmarks to register the device to the initial model. Examples of such systems are disclosed in detail in U.S. patent application Ser. No. 13/107,562 (filed May 13, 2011) (disclosing “Medical System Providing Dynamic Registration of a Model of an Anatomic Structure for Image-Guided Surgery”) and PCT application number PCT/US2016/033596 (filed May 20, 2016) (disclosing “Systems and Methods of Registration for Image Guided Surgery”) which are incorporated by reference herein in their entirety.

Once registered, the rendered image of the elongate device or medical device can be updated as the operator navigates the device to various points in anatomy. The navigational guidance can include the treatment plan including treatment parameters determined during process 350, including, for example, indicators showing locations, sizes, number, pattern, and number of treatment zones within the model. In some embodiments, the indicators can be altered in color, transparency, hue, size, etc., as ablative energy is delivered to tissue, thus providing the user with visual feedback of energy delivery. In some embodiments, the change in appearance of the indicators can be based on time of energy delivery and/or power of energy delivery. In other embodiments, the change in appearance of the indicators can be based on an expected effectiveness of ablation based on duration of energy delivery, power, and tissue type. In some embodiments, impedance sensors integrated into an ablation probe can measure impedance of tissue and be used as a measure of ablation effectiveness which can be reflected by a change in appearance of the indicators. The model can indicate the location and depth of target tissue, indicators (such as arrows or lines) can be displayed to guide the user from a current position of the device to the target tissue, an indicator can alert the operator to begin ablation when the device is positioned at target tissue, power levels and power durations can be displayed and adjusted as tissue becomes more desiccated during ablation, instructions can be provided to apply more pressure to tissue for deeper ablation, etc. The model can visually update an appearance of diseased tissue as it becomes ablated by changing color, shade, transparency, or hue relative to healthy or un-ablated tissue.

In one embodiment, the medical procedure plan can provide for display of an indicator instructing deployment of a protective device or a protective substance prior to delivering ablative energy or treatment. In one example, a protective device or protective cooling substance can be deployed through a working channel of the elongate instrument, in the position determined during process 350. Navigational guidance can be provided to aid the operator in positioning of the elongate device during delivery, positioning, and deployment of the protective device.

The procedure plan may be executed in a manual, semi-manual, or automated manner according to the navigational guidance and treatment plan. For example, the ablation probe may be manually positioned by the operator but as the registered and localized ablation device is positioned within anatomy, the medical instrument system can automatically apply power at a power level and a duration as provided by the treatment plan when the ablation device is positioned at target tissue. In some embodiments, power may be automatically reduced or turned off if the system detects the ablation probe is close to sensitive areas. In some embodiments, if a sensitive area is disposed in only a portion of the circumference of a target area, then power can be delivered only to the opposite side of the target area to avoid the sensitive area. Endoscopic visualization may be used so that the user may stop the ablation and/or override the procedure. In some embodiments, image-based recognition may be used to automatically identify when the ablation device has reached target tissue, when ablation is completed, or if the ablation probe is too close to sensitive areas.

The processes shown in the flowchart of medical procedure 300 may include additional processes or be performed in any order needed. For example, in some embodiments, target areas of interest may be identified in process 330 before the model of anatomy is created in process 320. In some embodiments, an initial model in process 320 is made from a generic model of human anatomy or from data such as a CT scan, then the target areas of interest in process 330 would be identified during as the anatomy is explored, and then the initial model would be updated in a separate process. In some embodiments, the model in process 320 is made while identifying target areas of interest in process 330. Additionally, in some embodiments, additional processes are performed. In some embodiments, the medical procedure performed is used to update the model. For example, the model may be updated with locations of ablated target tissue. Thus after an initial treatment performed in process 360, the procedure can return to process 340 where the location, size, and depth of ablated tissue is displayed on the current anatomical model to create an updated model which can be used to create an updated treatment plan requiring additional ablations.

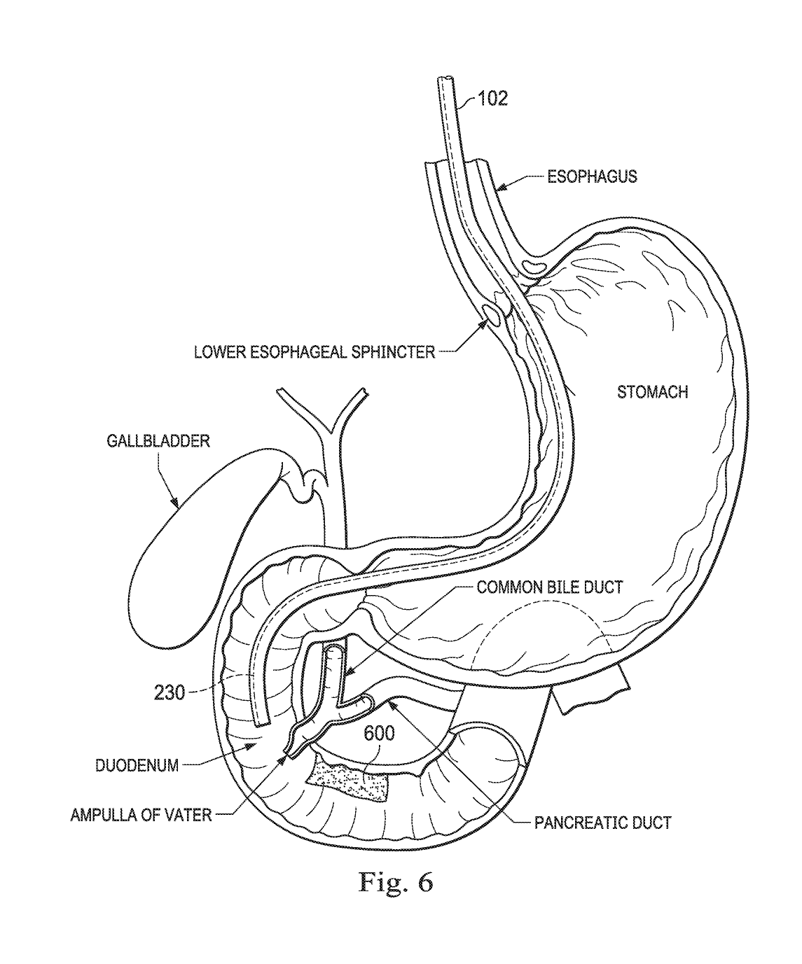

In one example, the systems and methods described above, with regards to medical procedure 300, can be applied to mapping and treatment of a patient's gastro-intestinal tract. FIG. 5 illustrates a representation of a portion of a patient digestive system including the stomach which leads to the small intestine. The small intestine includes three sections, a duodenum, jejunum, and ileum. The duodenum includes several layers of tissue including an innermost mucosal layer, which covers a submucosal layer, and muscle layers found deeper within the intestinal wall. Cells within the mucosal layer of the duodenum produce one or more hormones that impacts insulin production. When the mucosal cells become hyperplastic, an overabundance of hormones are produced which affects insulin secretion. The resulting impact on insulin secretion can result in insulin resistance and Type II diabetes. It is possible to treat the hyperplastic mucosal cells within the duodenum to treat insulin resistance. For example, all or a portion of the duodenal mucosal tissue can be ablated to slough off the hyperplastic mucosal cells. After ablation, the submucosa can generate replacement mucosal cells unaffected by hyperplasia, restoring normal insulin secretion.

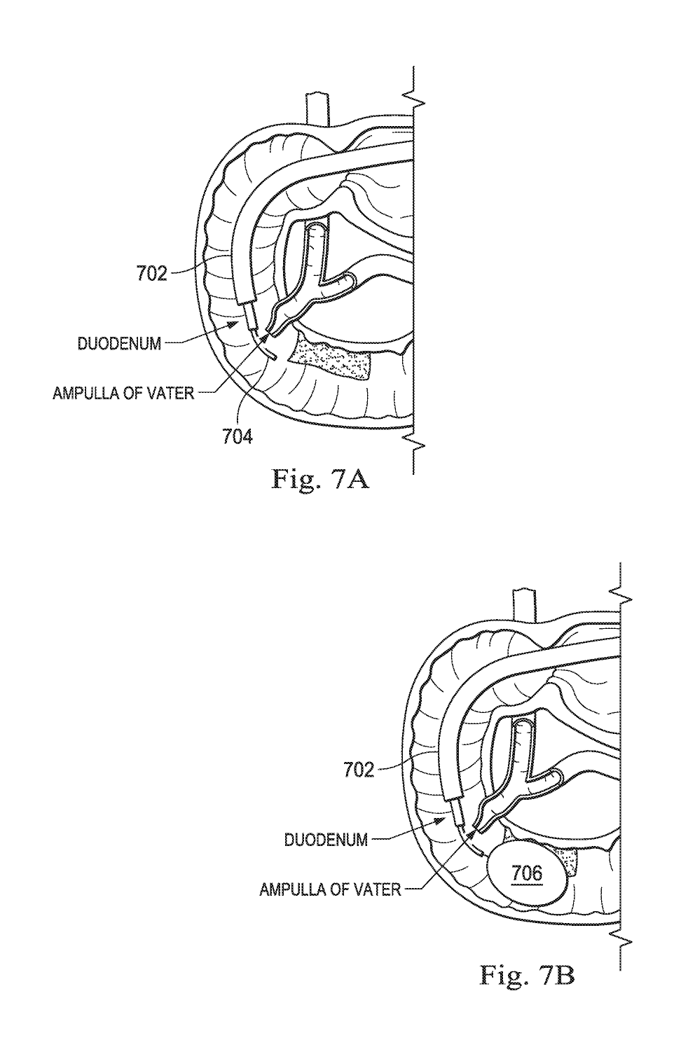

In some current medical procedures, a balloon catheter ablation device is endoluminally delivered over a guide wire through a patient esophagus and stomach into the duodenum for ablation of hyperplastic, neoplastic, dysplastic, diseased, or other tissue along a length of the duodenum. Yet current procedures can suffer from some drawbacks and challenges. Because the lining of internal passageways and hollow viscus structures are soft and distensible, areas such as the lining of the esophagus, stomach, and small intestine (e.g. the duodenum, biliary tree, pancreatic ducts, colon, and sinuses (e.g. front, maxillary, sphenoid and ethmoid)) can be damaged or perforated by the tip of the guide wire or an instrument. Additionally, positioning of the device near target tissue is commonly performed under fluoroscopy which often does not display hyperplastic tissue so that the ablation is performed blind additionally exposing the user and patient to radiation. While some procedures can be performed under endoscopic visualization where the hyperplastic tissue can be visible, the operator must avoid areas of sensitivity such as areas near the union of the pancreatic duct and the common bile duct, e.g. the ampulla of Vater (hepatopancreatic ampulla) and the papilla of Vater (major duodenal papilla) as can be seen in FIG. 6, which can be difficult to visualize especially when endoscopic views are compromised by debris.

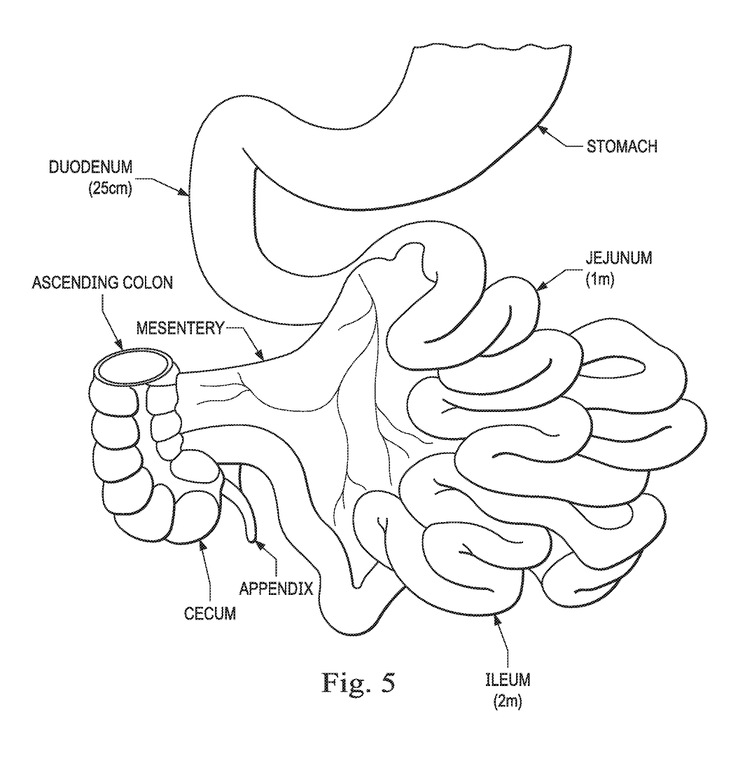

Referring to FIG. 3 and FIG. 4, an example of applying medical procedure 300 to a patient digestive system is provided including generating a model of the gastro-intestinal tract of a patient anatomy. The model may be used to plan areas of treatment, and/or used for treatment of hyperplastic tissue within the duodenum. For example, the elongate device 102 can be used to access and create a model of portions of the small intestine such as the duodenum, the jejunum, and/or the ileum. In one example, the elongate device 102 can be navigated to the duodenum as illustrated in FIG. 4. At process 310, the elongate device 102 can be inserted into a patient's mouth and navigated through the patient digestive tract including through the esophagus, through the gastroesophageal junction, through the stomach, and through the small intestine to an anatomic target. In an alternative embodiment (not shown), an external surgical incision could be made to provide direct access to the stomach and the elongate device can be inserted into the duodenum from the stomach. The more direct access approach directly from the stomach, would allow for shorter elongate devices to be used during a procedure.

Referring to process 320, an anatomic model, or map of the gastro-intestinal tract can be created. As previously described, the gastro-intestinal model may be generated from data collected using localization sensors (e.g. fiber optic shape and/or a plurality of EM sensors) coupled to the elongate device 102, as the elongated device 102 is navigated through the digestive tract, and well as information from imaging sensor 230 disposed through elongate device 102 or near a distal end of elongate device 102. In some embodiments, a processor such as control system may collect position and/or localization data representing the distal end 118 of the elongate device 102, thereby providing a centerline of the gastrointestinal tract. In some embodiments, the centerline would represent a path through the esophagus, through the stomach, and into the small intestine. In some embodiments, once the elongate device 102 is navigated to the target hyperplastic tissue, shape sensor data measuring the full shape of the elongated device 102 could be utilized to generate an instant centerline of the full gastro-intestinal tract or supplement the centerline model generated during collection of points representing the distal end 118 of the elongate device 102. The model may then be built including diameters of the esophagus, walls of the stomach, and walls of the duodenum by using either standard anatomical human data, using elongate device 102 and localization sensing to touch anatomical walls to determine volume, and/or data from imaging sensor 230 where diameter can be determined using image-based methods.

FIG. 6 illustrates a representation of patient anatomy including the esophagus, stomach and duodenum. Within the duodenum, the papilla of Vater forms a protrusion adjacent the ampulla of Vater which is illustrated connecting the pancreatic duct and the common bile duct. As shown in FIG. 6, hyperplastic tissue 600 can be located near structures such as the papilla of Vater/ampulla of Vater. During ablation of tissue, damage to the papilla of Vater and ampulla of Vater can be detrimental to patient health causing conditions such as pancreatitis. So it can be important to identify structures such as the ampulla of Vater as sensitive anatomy to avoid during ablative treatment. Thus, referring again to process 330, identifying target areas of interest can include anatomical target areas, such as areas identified for treatment such as hyperplastic tissue; structures or anatomy to be avoided during treatment such as the ampulla of Vater (as shown in FIG. 6) which may be located in close proximity to the treatment areas; and/or artificial locations such as a previously placed markers within the tissue. Hyperplastic or other target tissue and various anatomical structures (papilla of Vater, ampulla of Vater, etc.) can be identified visually in endoscopic camera images as well as in, e.g., ultrasound or OCT images. In some embodiments, such landmarks can be user identified where the operator may signal the location of the landmark by press a button when the elongate device is adjacent to or touching a landmark. In some embodiments, ultrasound may be used to determine depth of hyperplastic tissue. With localization sensors provided within the elongate device, an exact location of the hyperplastic tissue correlated to depth of the hyperplastic tissue can be determined.

Referring back to process 340, the target areas of interest identified in process 330 may then be displayed or rendered on the model of the anatomy generated in process 320 as previously described. The target areas of interest can include hyperplastic tissue, the papilla of Vater, and the ampulla of Vater. In some embodiments, if ultrasound is used to identify depth of hyperplastic tissue, the hyperplastic tissue can be displayed at a measured location within the model, at a detected depth based on ultrasound data, and in a different color, shade, hue, or transparency from normal tissue.

Referring to process 350, the model may be used to plan a medical procedure for treatment of hyperplastic tissue including determining a navigational path to the hyperplastic tissue and/or creating a treatment plan. The navigational path can be established within the model by the user by manually identifying a path through a patient's mouth, through the esophagus, through the gastroesophageal junction, through the stomach, and into small intestine. In other embodiments, the computer processor can automatically generate a path through anatomy to the hyperplastic tissue within the small intestine. As previously described, the treatment plan can be automatically or manually created to identify treatment parameters such as treatment location, size of treatment area, treatment depth, number of treatments, and spacing of treatments. Sensitive areas to avoid within anatomy such as the papilla of Vater/ampulla of Vater can be automatically or manually identified and power levels can be automatically or manually set and saved within the procedure plan to reduce power near the sensitive anatomy, and increase power levels at locations with hyperplastic tissue. Additionally the sensitive areas can be identified as locations of deployment for protective devices and/or protective substances.

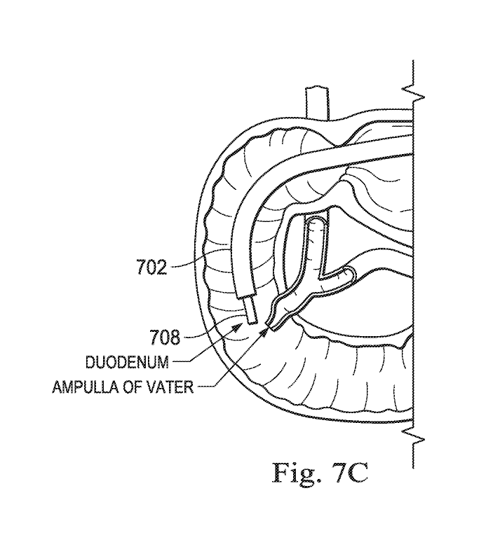

Using the medical procedure plan determined in process 350, a treatment procedure for hyperplastic tissue can be performed in process 360. FIGS. 7a through 7c illustrate a portion of a patient small intestine including the duodenum, the papilla of Vater/ampulla of Vater, and hyperplastic target tissue. In one example, the treatment procedure of process 360 begins with an elongated device 702 (such as device 102, 202) positioned within the target anatomy as illustrated in FIG. 7a such that the elongate device 702 is positioned near the target hyperplastic tissue. The elongate device can include a working lumen for delivery of tools and devices required for the medical procedure. In some examples, the medical procedure plan includes deploying a protective device or protective substance. For example, the working lumen, or a separate delivery device can be deployed through the working lumen, may be used to deliver saline or water to cool areas surrounding the target ablation area. In another example, a sheath 704 can be deployed through the working lumen of the elongate device, over the papilla of Vater and ampulla of Vater. The treatment device can then be deployed through that sheath, protecting the papilla of Vater and ampulla of Vater when treatment energy is delivered.

Methods of treatment can include expanding the submucosal layer such as by fluid injection to flatten it and using a balloon 706 filled with hot air or water to ablate the tissue as illustrated in FIG. 7b. The contact balloon treatment could also be performed by using radio frequency (RF), ultrasound, microwave with a fluid filled balloon. A second class of treatments (not shown) rely on plugging a section of duodenum to be treated and then applying the treatment methodology such as flowing gas, heated vapor, or applying chemical such as alcohol. A third class of treatments are more targeted ablation therapies including RF, microwave, ultrasound, direct heat, laser ablation, cryo-abation, etc. delivered by an ablative medical instrument 708 as shown in FIG. 7c may be used to target specific areas of hyperplastic tissue. Such treatments as laser, sprays may be used for shallow penetration, thus obviating the need for injection of fluid first into the submucosal layer. Finally, mechanical devices such as a physical scraper or resecting device could be used

The treatment plan can include not only locations, sizes, and depth of hyperplastic tissue, but also margins around hyperplastic tissue at a lesser depth than where the hyperplastic tissue was visibly initially identified during the identification stage. During the delivery of ablative energy, hyperplastic or other target tissue and various anatomical structures can be identified visually in endoscopic camera images as well as in, e.g., ultrasound or OCT images. Tissue may be identified from as ablated or non-ablated tissue by measuring impedance of the tissue and monitoring a change in impedance or comparing impedance measurements against pre-determined impedance thresholds. The operator can deviate from the medical plan saved during process 350, and deviate from location and power delivery based on live endoscopic images. In some embodiments, power can automatically be reduced and or turned off based on proximity to sensitive areas established within the medical plan. In some embodiments, if a sensitive area is disposed in only a portion of the circumference of a target area, such as the papilla of Vater and ampulla of Vater, then power can be delivered only to the opposite side of the target area to avoid the sensitive area.

In some examples of medical procedures, within a body lumen such as the gastrointestinal tract, the medical device may be used as a diagnostic or treatment device for insulin resistance as will be described in detail below. In another example, the medical device may be routed to the esophagus to ablate maladies such as esophageal cancer. In another example, the medical device can be routed through a patient trachea into branches of patient airways to model patient lungs for mucosal resurfacing or ablation for treatment of lung conditions. Individual airway branches can be marked visually during navigation and treatment overlaps can be determined during a procedure such as for treatments for chronic obstructive bronchitis or asthma (for example, using RFA, cryospray, microwave, electroporation, etc.). In yet another example, the medical device can be used for urology cases to real time map the chambers of the kidney. Initially, the medical device can be used to explore all the chambers of the kidney including endoscopically, visually determining the location and size of kidney stones. A model of the kidneys can then be created including mapping the location and size of the kidney stones. The medical procedure can include using the medical device, or delivering a tool through the medical device, to break the kidney stones. After stone breakage, the user can deliver the medical device to the different calyxes to visually examine stone fragments remaining from the stone breakage procedure. The location and size of the remaining stone fragments can then we used to update the model of the kidney and could enable all the kidney chambers and remaining stones be tracked. Mapping and modeling could be particularly useful because not all patients with kidney stones have a CT taken and not all with have a pre-op image/map. Thus the map created during the medical procedure could be the only navigational guide provided to a user.

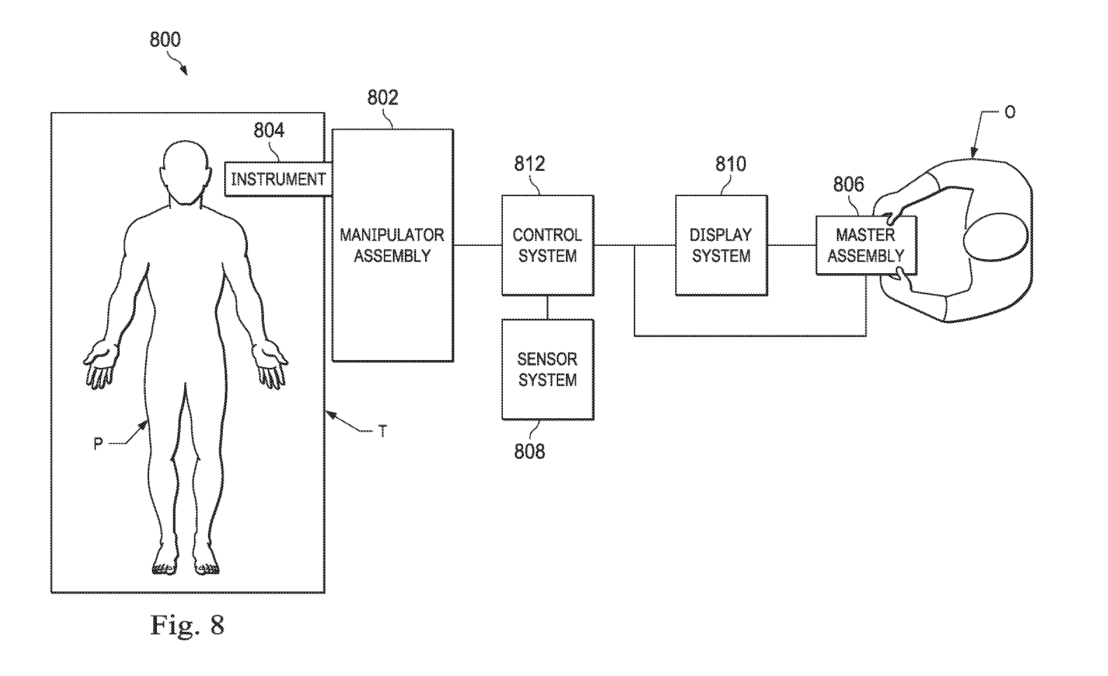

In some embodiments, the medical instrument 226 may be an ablation probe and may be robotically controlled so an ablation pattern may be robotically created according to the treatment plan. In some embodiments, the navigation of the ablation probe can be fully automated from entry into patient anatomy, navigation of the ablation probe to target tissue, and during ablation according to the navigation and treatment plans. FIG. 8 is a simplified diagram of a teleoperated medical system 800 which may be used to robotically control instruments 804, such as ablation probes, medical instruments, elongate devices, and/or the like. In some embodiments, teleoperated medical system 800 may be suitable for use in, for example, surgical, diagnostic, therapeutic, ablative or biopsy procedures described herein.

As shown in FIG. 8, medical system 800 generally includes a manipulator assembly 802 for operating a medical instrument 804 in performing various procedures on a patient P. The manipulator assembly 802 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 806 allows an operator O (e.g., a user, a surgeon, a clinician, and/or a physician as illustrated in FIG. 8) to control manipulator assembly 802. A display system 810 (e.g., the display system 110) allows the operator to view the interventional site, by displaying a live image and/or a representation of the surgical site and medical instrument 804 generated by sub-systems of sensor system 808, as well as displaying images and instructions for navigational guidance. Sensor system 808 can include one or more sub-systems for receiving information about the instruments of manipulator assembly 802. The sensor system 808 may include sensors 106, 108, for example. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that may make up medical instrument 804; and/or a visualization system for capturing images from the distal end of medical instrument 804. Teleoperated medical system 800 may also include control system 812 (e.g., the control system 122). Control system 812 includes at least one memory and at least one computer processor (not shown) for effecting control between medical instrument 804, master assembly 806, sensor system 808, and display system 810. Control system 812 also includes programmed instructions (e.g., a non-transitory machine-readable medium storing the instructions) to implement some or all of the methods described in accordance with aspects disclosed herein, including instructions for providing information to display system 810. In some embodiments, control system 812 may receive force and/or torque feedback from force sensors in the medical instrument 804.

One or more elements in embodiments of the invention may be implemented in software to execute on a processor of a computer system such as control system 112/812. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

What is claimed is:

1. A method performed by a processor, for accessing and treating a target tissue within a patient anatomy, the method comprising:
creating a first model of the patient anatomy using information from at least one localization sensor and at least one imaging sensor, wherein the at least one localization sensor and the at least one imaging sensor are coupled to a medical device being delivered through the patient anatomy to the target tissue;
receiving data identifying at least one anatomical area of interest within the patient anatomy;
creating a second model based on the first model and the at least one identified anatomical area of interest;
displaying the second model; and
performing a medical procedure on at least one of the anatomical areas of interest.

2. The method of claim 1, wherein the first model of the patient anatomy is further created using the information received from the at least one localization sensor or the at least one imaging sensor when the medical device is touching a side of a body lumen, wherein the information is used to size the body lumen.

3. The method of claim 1, wherein the at least one anatomical area of interest is detected using the imaging sensor.

4. The method of claim 1, wherein the data identifying the at least one anatomical area of interest further comprises receiving information from a user.

5. The method of claim 1, further comprising generating a medical plan based the second model, wherein the medical plan includes a treatment plan.

6. The method of claim 5, wherein the anatomical areas of interest includes the target tissue and at least one sensitive anatomical area.

7. The method of claim 6, wherein the treatment plan comprises:
ablating the target tissue; and
protecting the at least one sensitive anatomical area from ablation energy.

8. The method of claim 7, wherein protecting the at least one sensitive anatomical area from ablation energy includes setting a level of ablation energy based on proximity to the at least one sensitive anatomical area or delivering a device to protect the at least one of the anatomical areas of interest.

9. The method of claim 7, wherein ablating the target tissue includes setting a level of ablation energy based on a depth of the target tissue.

10. The method of claim 1, wherein the medical procedure includes treatment using one or more of direct heat, laser, gas, radio frequency, microwave, ultrasound, cryotherapy, scraping, or vapor.

11. The method of claim 1, wherein the patient anatomy is a gastrointestional tract and the target tissue is within a duodenum.

12. The method of claim 11, wherein performing the medical procedure further comprising delivering a needle through channel of the medical device to deliver fluid behind a mucosal lining of the duodenum.

13. The method of claim 1, wherein the anatomical areas of interest comprises at least one of hyperplastic tissue, neoplastic tissue, dysplastic tissue, diseased tissue, a tumor, a nodule, or an ampulla of Vater.

14. A medical system for accessing a patient anatomy, the medical system comprising:
a medical device including a main lumen;
a localization sensor providing position of the medical device;
an imaging sensor configurable to provide an image from a distal portion of the medical device;
a processor in communication with the localization sensor and the imaging sensor, wherein the processor is configured to:
generate a first model using information from the localization sensor and the imaging sensor;
receive data for identifying anatomic features of interest within the patient anatomy;
update the first model to generate a second model including the anatomic features of interest; and
a display for displaying at least one of the first model and the second model.

15. The medical system of claim 14, wherein the processor is further configured to generate a medical plan based on the second model and generate indicators for guidance to perform a medical procedure based on the medical plan.

16. The medical system of claim 14, wherein the localization sensor is at least one of a fiber optic sensor, an EM sensor, or a vision probe.

17. The medical system of claim 14, wherein the imaging sensor is integrated into the medical device or the imaging sensor is disposed through a lumen of the medical device.

18. The medical system of claim 14, wherein the medical device is flexible and steerable and the localization sensor is integrated into the medical device.

19. The medical system of claim 14, further comprising a sheath and wherein the localization sensor and the imaging sensor are each integrated into to the sheath.

20. The medical system of claim 14, the medical system further comprising a force sensor configurable to provide force feedback from the distal portion of the medical device.
